(19) United States
(12) Patent Application Publication
(10) Pub. No.: US 2020/0377518 A1
(43) Pub. Date: Dec. 3, 2020

(54) METHOD OF PREVENTING AND TREATING THROMBOSIS

(71) Applicant: Rigel Pharmaceuticals, Inc., South San Francisco, CA (US)

(72) Inventor: Esteban Masuda, Menlo Park, CA (US)

(73) Assignee: Rigel Pharmaceuticals, Inc., South San Francisco, CA (US)

(21) Appl. No.: 16/888,430

(22) Filed: May 29, 2020

Related U.S. Application Data
(60) Provisional application No. 62/853,937, filed on May 29, 2019.

(51) Int. Cl. C07D 498/04; A61P 7/02

(52) U.S. Cl. CPC C07D 498/04; A61P 7/02

(57) ABSTRACT

A method of preventing or treating thrombosis in a patent is disclose, wherein the method comprises administering to the patient an amount of fostamatinib or a form or metabolite thereof effective to prevent or treat the thrombosis, respectively.

### CROSS REFERENCE TO RELATED APPLICATION

This application claims the benefit of the earlier filing date of U.S. Provisional Application No. 62/853,937, filed on May 29, 2019, the entirety of which is incorporated herein by reference.

### FIELD

The invention is in the field of treatment and prevention of thrombosis with small organic molecule inhibitors.

### SUMMARY OF THE RELATED ART

The FDA has approved TAVALISSE® for the treatment of thrombocytopenia in adult patients with immune thrombocytopenia (ITP) who have had an insufficient response to a previous treatment. The active agent in TAVALISSE® is fostamatinib disodium hexahydrate, [6-[[5-fluoro-2-(3,4,5-trimethoxyanilino)pyrimidin-4-yl]amino]-2,2-dimethyl-3-oxopyrido[3,2-b][1,4]oxazin-4-yl]methyl phosphate disodium hexahydrate,

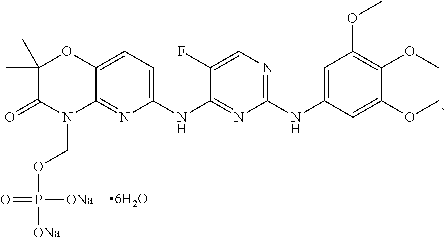

the disodium hexahydrate of fostamatinib (R788), [6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate,

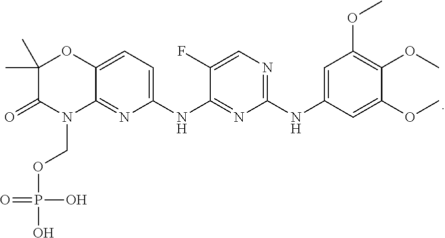

Fostamatinib and its disodium hexahydrate salt are prodrugs of the active metabolite 6-((5-fluoro-2-((3,4,5-trimethoxyphenyl)amino)pyrimidin-4-yl)amino)-2,2-dimethyl-2H-pyrido[3,2-b][1,4]oxazin-3(4H)-one (R406),

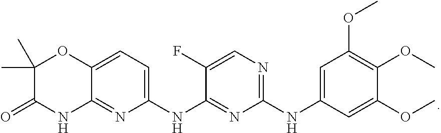

R406 is an inhibitor of the enzyme spleen tyrosine kinase (Syk), which plays a key role in the signaling of activating Fc receptors and the B-cell receptor (BCR). Syk is involved in the signal transduction pathways associated with the high affinity Fc receptors for IgE (“FcεRI”) and/or IgG (“FcγRI”) (see Valent et al., 2002, Intl. J. Hematol. 75(4):257-362 for review). Biochemical data confirm that 2,4-pyrimidinediamine compounds such as R406 exert degranulation inhibitory effect, at least in part, by blocking or inhibiting the signal transduction cascade(s) initiated by crosslinking of FcεRI and/or FcγRI (see, e.g., U.S. application Ser. No. 10/631,029 filed Jul. 29, 2003 (US2007/0060603, now U.S. Pat. No. 7,517,886), international application Ser. No. PCT/US03/24087 (WO2004/014382), U.S. application Ser. No. 10/903,263 filed Jul. 30, 2004 (US2005/0234049), and international application Ser. No. PCT/US2004/24716 (WO/2005/016893), the disclosures of which are incorporated herein by reference.

Braselmann et al. (J. Pharmacol. Exp. Ther. 319(3): 998-1008 (December 2006; epub Aug. 31, 2006) demonstrated that R406 is a potent inhibitor of IgE- and IgG-mediated activation of Fc receptor signaling (EC₅₀ for degranulation=56-64 nM). R406 inhibited phosphorylation of Syk substrate linker for activation of T cells in mast cells and B-cell linker protein/SLP65 in B cells. R406 bound to the ATP binding pocket of Syk and inhibited its kinase activity as an ATP-competitive inhibitor (K(i)=30 nM). Furthermore, R406 blocked Syk-dependent FcR-mediated activation of monocytes/macrophages and neutrophils and BCR-mediated activation of B lymphocytes.

### SUMMARY OF THE INVENTION

Surprisingly, we have found that although fostamatinib increases platelet levels in immune thrombocytic purpura (ITP) patients and thrombosis is associated with an excess of platelets, fostamatinib does not appear to increase risk of thrombosis. Further, we have found that fostamatinib can combat thrombosis, which, without being limited to any particular theory, we believe results from fostamatinib blocking Fc receptor signaling. Accordingly, the present invention comprises a method of preventing or treating thrombosis in a patient using a form of fostamatinib or its active metabolite, R406.

### DETAILED DESCRIPTION OF THE INVENTION

The present invention comprises a method of prevention of thrombosis in a patient (particularly a patient having one or more risk factors that make the patient have a high risk of developing thrombosis relative to the general population), the method comprising administering to the patient an amount of a form of fostamatinib effective to prevent thrombosis in the patient.

The present invention also comprises a method of treating thrombosis in a patient having a thrombus, the method comprising administering to the patient an amount of a form of fostamatinib effective to treat the thrombosis in the patient.

Thrombosis is viewed as a clotting disorder to which an excess of platelets contributes. Consistent with this view is the fact that chronic ITP patients treated with agents, such as the thrombopoietin (TPO) agonists eltrombopag and romiplostim, that upregulate platelet formation are at increased risk for thrombosis (e.g., Med Clin (Barc). 2015 Dec. 21; 145(12):511-9, Epub 2015 Jun. 4, and Dig Liver Dis. 2019 January; 51(1):24-27, Epub 2018 Jun. 20.) In addition to risk of thrombosis introduced by TPO agonists, ITP itself may be considered a risk factor (Takagi, et al., J Hematol Thrombo Dis 2015, 3:1). Takagi propose that thrombosis is a “comorbidity that may require special attention as both management of thromboembolism and thrombophylaxis in patients with low platelet levels can be challenging.” Indeed, balancing of platelet destruction with platelet production and maintenance requires careful management of ITP interventions. Many of the standard treatments for ITP, including corticosteroids, such as prednisone, IVIG, splenectomy, TPO agonists, introduce additional risk of thromboembolism. Accordingly, both untreated and treated ITP patients are at risk for thrombosis and can be treated as disclosed herein with fostamatinib, R406 or both to prevent or ameliorate thrombosis.

In one embodiment of the method of prevention, the patient is at heightened risk relative to the general population (e.g., as measured by recognized risk factors) of a thrombolytic event. Risk factors for thrombosis include, in addition to those associated with ITP and its treatment, include those discussed in Previtali, Blood Transfus 9:120-38 (2011), such as classical cardiovascular disease risk factors: hyperlipidemia, smoking, diabetes, hypertension, and abdominal obesity; strong classical venous thromboembolism risk factors: trauma or fractures, major orthopedic surgery, and oncological surgery; moderate classical venous thromboembolism risk factors: non-oncological surgery, oral contraceptives and hormone replacement therapy, pregnancy and puerperium, hypercoagulability, and previous venous thromboembolism; and weak classical venous thromboembolism risk factors: age, bed rest (>3 days), prolonged travel, and metabolic syndrome. Additional risk factors include inherited, acquired and mixed coagulation or metabolic risk factors for thrombosis:

| Inherited | Acquired | Mixed |
|---|---|---|
| Antithrombin | Antiphospholipid | Hyperhomocysteinaemia |
| deficiency | syndrome | |
| Protein C deficiency | | Increased fibrinogen levels |
| Protein S deficiency | | Increased factor VIII levels |
| Factor V Leiden | | Increased factor IX levels |
| Prothrombin G20210A | | |

Heparin is a naturally occurring mucopolysaccharide with a molecular size of 5000-25,000 daltons. Heparin for human use is typically derived from bovine or porcine sources and is used in its unfractionated form or in a fractionated form. Unfractionated heparin is a mucopolysaccharide with an average molecular weight of 15,000-18,000 daltons. Heparin acts by binding reversibly to antithrombin III, accelerating its action on coagulation factors XII, XI, X, IX, plasmin, and thrombin. It also inhibits platelet activation by fibrin.

Heparin is one of the most effective and widely used anticoagulants in hospitalized patients with cardiovascular diseases. Although hemorrhagic events are the main recognized risk of heparin use, heparin-induced thrombocytopenia (HIT) is a potentially severe, morbid complication of heparin therapy. It is common in practice and its most important consequence is a paradoxical increase in the risk of clotting (thromboembolic) complications. Several factors are thought to influence the frequency of HIT, including the type of heparin and the type of patient, with patients who have had a surgery at higher risk.

HIT is generally defined as a relative reduction in platelet count of about 50% (even if the platelet count at its lowest remains greater than 150×10⁹/L) occurring within five to 14 days after the start of heparin therapy. Subjects re-exposed after a recent treatment may develop a rapid onset of HIT within 24 hours of heparin administration. A less common presentation of HIT is a delayed onset following discontinuation of heparin. Formerly designated white clot syndrome or HIT type II, it is considered an acquired hypercoagulability syndrome caused by an immune-mediated reaction which is commonly followed by venous or arterial thrombosis. About 30% to 70% of untreated HIT patients develop venous or arterial thrombi that are life-/limb-threatening.

Unfractionated heparin (UFH) is administered parenterally, both subcutaneously for its prophylaxis and as a continuous intravenous infusion when used therapeutically. Intravenous heparin is usually given as a bolus of 100 U/kg followed by approximately 1000 U/h titrated to achieve an activated partial thromboplastin time (aPTT) of 1.5-2.5 times the control.

Several varieties of low molecular weight heparins have been developed. As used in the present invention these products will be treated as a class although they have different formulations and are not strictly interchangeable clinically. See, e.g., Maddineni J, Walenga J M, Jeske W P, et al. Product individuality of commercially available low-molecular-weight heparins and their generic versions: Therapeutic implications. Clin Appl Thromb Hemost. 2006; 12:267-276.

Examples of low molecular weight heparin for use in the present invention include, without limitation, enoxaparin (sold under the trade name Lovenox™) dalteparin (sold under the trade name Fragmin™) nadroparin, certoparin and tinzaparin (sold under the trade name Innohep™). Generic and bioequivalent versions of low molecular weight heparins also are known to those of ordinary skill in the art and are commercially available.

Low molecular weight heparin has an average molecular weight of 2000-10,000 daltons with a greater ability to inhibit factor Xa, than thrombin. See, Oranmore-Brown C, Griffiths R. Anticoagulants and the perioperative period. Contin Educ Anaesth Crit Care Pain 2006; 6:156-9. It has a more predictable dose response curve than unfractionated high molecular weight heparin and is administered at fixed dose, based on total body weight.

Low molecular weight heparin is indicated for thromboprophylaxis and treatment of deep vein thrombosis (DVT) and pulmonary embolism, myocardial infarction. Low molecular weight heparin has been demonstrated to be efficacious as a bridge therapy for patients anticoagulated with warfarin including parturients, patients with prosthetic heart valves, or preexisting hypercoagulable condition. In one embodiment a subject in need thereof is administered fostamatinib, its active metabolite (R406) or both, in conjunction with low molecular weight heparin to treat or prevent thrombosis and/or myocardial infarction. In an optional aspect of this method, the subject is identified as being at risk for thrombosis. Such subjects are identified as is described herein as those who, have, for example received high molecular weight heparin. In another aspect of the disclosed method, the subject has been treated with unfractionated heparin during surgery and is treated with fostamatinib, R406 or with optional low molecular weight heparin in the post-operative recovery period.

Patients undergoing post- and perioperative surgical care are at increased risk for heparin-induced thrombocytopenia (HIT). In particular, subjects who have undergone cardiovascular interventions, including those with stable coronary artery disease (CAD) and acute coronary syndromes (ACSs) are at risk as they are likely to have received unfractionated heparin. Such, high molecular weight heparin is more likely to be used in this context and is believed to contribute more to thrombotic risk than low molecular weight heparin that may be used in other treatment contexts. Accordingly, in a particular embodiment of the present method of preventing thrombosis, the patient is one who was treated with heparin, particularly high molecular weight heparin or unfractionated heparin, following surgery.

Zhou et al. (Blood, December 2015, volume 126, number 26, p. 2817; DOI 10.1182/blood-2015-02-631135) demonstrated that anti-miRNA could be used to regulate platelet reactivity in mice in vivo and by upregulating the histidine tyrosine phosphatase, TULA-2. Zhou et al. further suggested that the anti-sense approach could be potentially translated to humans because the sequence of the miRNA targeted, miR-148a, and its binding site on TULA-2 are highly conserved between mice and humans.

As used herein, “a form of fostamatinib” means fostamatinib, a pharmaceutically acceptable salt thereof, a pharmaceutically acceptable salt hydrate thereof, or a Syk-inhibiting metabolite thereof. In one embodiment, the form of fostamatinib is fostamatinib disodium hexahydrate (e.g., TAVALISSE®). In another embodiment, the form of fostamatinib is the metabolite R406.

The form of fostamatinib can be administered alone or in a pharmaceutical composition manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically.

The form of fostamatinib or R406 and pharmaceutical formulations comprising these compounds can be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients and vehicles appropriate for each route of administration.

Pharmaceutical compositions comprising a form of fostamatinib may conveniently be presented in dosage unit form and can be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired therapeutic effect. For example, pharmaceutical compositions of the invention may take a form suitable for virtually any mode of administration, including, for example, topical, ocular, oral, buccal, systemic, nasal, injection, transdermal, rectal, vaginal, etc., or a form suitable for administration by inhalation or insufflation.

For topical use, creams, ointments, jellies, gels, solutions or suspensions, etc., containing a form of fostamatinib can be employed. In certain embodiments, a form of fostamatinib can be formulated for topical administration with polyethylene glycol (PEG). These formulations may optionally comprise additional pharmaceutically acceptable ingredients such as diluents, stabilizers and/or adjuvants.

According to the invention, a form of fostamatinib can be used for manufacturing a composition or medicament for use in the prevention or treatment of thrombosis in a patient having a heightened risk of developing thrombosis or a suffering from thrombosis, respectively. The composition or medicament can be formulated for delivery by any of the means disclosed herein.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, dextrose solution, etc., before use. To this end, the active compound(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars, films or enteric coatings. Additionally, the pharmaceutical compositions containing the fostamatinib as active ingredient or prodrug thereof in a form suitable for oral use, may also include, for example, troches, lozenges, aqueous or oily suspensions, dispersible powders or granules, emulsions, hard or soft capsules, or syrups or elixirs. Compositions intended for oral use can be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient (including prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch, or alginic acid); binding agents (e.g. starch, gelatin or acacia); and lubricating agents (e.g. magnesium stearate, stearic acid or talc). The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques described in the U.S. Pat. Nos. 4,256,108; 4,166,452; and U.S. Pat. No. 4,265,874 to form osmotic therapeutic tablets for control release. The pharmaceutical compositions of the invention may also be in the form of oil-in-water emulsions.

Liquid preparations for oral administration may take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration can be suitably formulated to give controlled release of the active compound or prodrug, as is well known.

For buccal administration, the compositions may take the form of tablets or lozenges formulated in conventional manner.

For rectal and vaginal routes of administration, the active compound(s) can be formulated as solutions (for retention enemas) suppositories or ointments containing conventional suppository bases such as cocoa butter or other glycerides.

For nasal administration or administration by inhalation or insufflation, the active can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that can be employed are water, Ringer's solution and isotonic sodium chloride solution.

According to the present invention, a form of fostamatinib can also be delivered by any of a variety of inhalation devices and methods known in the art, including, for example: U.S. Pat. Nos. 6,241,969; 6,060,069; 6,238,647; 6,335,316; 5,364,838; 5,672,581; WO96/32149; WO95/24183; U.S. Pat. Nos. 5,654,007; 5,404,871; 5,672,581; 5,743,250; 5,419,315; 5,558,085; WO98/33480; U.S. Pat. Nos. 5,364,833; 5,320,094; 5,780,014; 5,658,878; 5,518,998; 5,506,203; 5,661,130; 5,655,523; 5,645,051; 5,622,166; 5,577,497; 5,492,112; 5,327,883; 5,277,195; U.S. Publication No. 20010041190; U.S. Publication No. 20020006901; and U.S. Publication No. 20020034477.

Included among the devices which can be used to administer a form of fostamatinib are those well-known in the art, such as, metered dose inhalers, liquid nebulizers, dry powder inhalers, sprayers, thermal vaporizers, and the like. Other suitable technology for administration of particular 2,4-pyrimidinediamine compounds includes electrohydrodynamic aerosolizers.

In addition, the inhalation device is preferably practical, in the sense of being easy to use, small enough to carry conveniently, capable of providing multiple doses, and durable. Some specific examples of commercially available inhalation devices are Turbohaler (Astra, Wilmington, Del.), Rotahaler (Glaxo, Research Triangle Park, N.C.), Diskus (Glaxo, Research Triangle Park, N.C.), the Ultravent nebulizer (Mallinckrodt), the Acorn II nebulizer (Marquest Medical Products, Totowa, N.J.) the Ventolin metered dose inhaler (Glaxo, Research Triangle Park, N.C.), or the like. In one embodiment, fostamatinib can be delivered by a dry powder inhaler or a sprayer.

As those skilled in the art will recognize, the formulation of the form of fostamatinib, the quantity of the formulation delivered, and the duration of administration of a single dose depend on the type of inhalation device employed as well as other factors. For some aerosol delivery systems, such as nebulizers, the frequency of administration and length of time for which the system is activated will depend mainly on the concentration of fostamatinib in the aerosol. For example, shorter periods of administration can be used at higher concentrations fostamatinib in the nebulizer solution. Devices such as metered dose inhalers can produce higher aerosol concentrations and can be operated for shorter periods to deliver the desired amount of fostamatinib in some embodiments. Devices such as dry powder inhalers deliver active agent until a given charge of agent is expelled from the device. In this type of inhaler, the amount of 2 fostamatinib in a given quantity of the powder determines the dose delivered in a single administration. The formulation of fostamatinib is selected to yield the desired particle size in the chosen inhalation device.

Formulations of a form of fostamatinib for administration from a dry powder inhaler may typically include a finely divided dry powder containing fostamatinib, but the powder can also include a bulking agent, buffer, carrier, excipient, another additive, or the like. Additives can be included in a dry powder formulation of fostamatinib, for example, to dilute the powder as required for delivery from the particular powder inhaler, to facilitate processing of the formulation, to provide advantageous powder properties to the formulation, to facilitate dispersion of the powder from the inhalation device, to stabilize to the formulation (e.g., antioxidants or buffers), to provide taste to the formulation, or the like. Typical additives include mono-, di-, and polysaccharides; sugar alcohols and other polyols, such as, for example, lactose, glucose, raffinose, melezitose, lactitol, maltitol, trehalose, sucrose, mannitol, starch, or combinations thereof; surfactants, such as sorbitols, diphosphatidyl choline, or lecithin; or the like.

The method of the invention can be conducted a pharmaceutical composition including a form of fostamatinib suitable for administration by inhalation. For example, a dry powder formulation can be manufactured in several ways, using conventional techniques, such as described in any of the publications mentioned above and incorporated expressly herein by reference, and for example, Baker, et al., U.S. Pat. No. 5,700,904, the entire disclosure of which is incorporated expressly herein by reference. Particles in the size range appropriate for maximal deposition in the lower respiratory tract can be made by micronizing, milling, or the like. And a liquid formulation can be manufactured by dissolving the fostamatinib in a suitable solvent, such as water, at an appropriate pH, including buffers or other excipients.

Pharmaceutical compositions comprising a form of fostamatinib can be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically. A wide variety of suitable pharmaceutical compositions are known in the art. See, e.g., Remington: The Science and Practice of Pharmacy, volume I and volume II. (22^{nd} Ed., University of the Sciences, Philadelphia).

Inactive ingredients include mannitol, sodium bicarbonate, sodium starch glycolate, povidone, and magnesium stearate, any or all of which, when the fostamatinib formulation is in table form, can be in the tablet core. Tablets may also be film coated, and the file coating can comprise one or more of polyvinyl alcohol, titanium dioxide, polyethylene glycol 3350, talc, iron oxide yellow, and iron oxide red.

For ocular administration, the fostamatinib can be formulated as a solution, emulsion, suspension, etc. suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851.

For prolonged delivery, the fostamatinib can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) may also be employed, although usually at the cost of greater toxicity.

In one embodiment, the form of fostamatinib is administered orally in the form of a tablet, e.g., TAVALISSE®.

The amount of a form of fostamatinib administered will depend upon a variety of factors, including, for example, the mode of administration, the severity of the condition being treated and the age and weight of the patient, etc. Determination of an effective dosage is well within the capabilities of those skilled in the art.

As known by those of skill in the art, the preferred dosage of the form of fostamatinib will also depend on the age, weight, general health and severity of the condition of the individual being treated. Dosage may also need to be tailored to the sex of the individual and/or where administered by inhalation, the lung capacity of the individual. Dosage may also be tailored to individuals suffering from more than one condition or those individuals who have additional conditions which affect lung capacity and the ability to breathe normally, for example, emphysema, bronchitis, pneumonia, respiratory infections, etc. Dosage, and frequency of administration of the compounds or prodrugs thereof, will also depend on whether the compounds are formulated for treatment of acute episodes of thrombocytopenia or for the prophylactic treatment of such a disorder. A skilled practitioner will be able to determine the optimal dose for a particular individual.

Effective dosages can be estimated initially from in vitro assays. For example, an initial dosage for use in animals can be formulated to achieve a circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans. For guidance, the reader is referred to Fingl & Woodbury, “General Principles,” In: Goodman and Gilman's The Pharmaceutical Basis of Therapeutics, Chapter 1, 13th edition, Pergamon Press, 2017, and the references cited therein.

Initial dosages can also be estimated from in vivo data, such as animal models. Animal models useful for testing the efficacy of compounds to treat or prevent the various diseases described above are well-known in the art.

Dosage amount and interval can be adjusted individually to provide plasma levels of the compound that is sufficient to maintain therapeutic effect. For example, the form of fostamatinib can be administered once per week, several times per week (e.g., every other day), once per day or multiple times per day, depending upon, among other things, the mode of administration, the specific indication being treated and the judgment of the prescribing physician. By way of example, in a subject at risk for thrombosis, the subject may be monitored using methods known to those of skill in the art of maintaining hemostasis in patients at risk for thrombosis, such as peri- or post-operative patients. Examples of methods for monitoring patients at risk of thrombosis included, without limitation, digital subtraction angiography, in vitro assays or non-invasive methods. Examples of in vitro assays useful for identifying and monitoring subjects at risk for thrombosis and for treatment using the present methods include, without limitation, functional assays and antibody detection assays. Functional assays are those that depend on detecting platelet activation following exposure of normal platelets to patient serum or plasma in the presence of heparin. Examples of such assays include platelet aggregation assays as described in Nguyen P, Lecompte T. Heparin-induced thrombocytopenia: a survey of tests employed and attitudes in haematology laboratories. Groupe d'Étude sur l'Hemostase et la Thrombose (GEHT) de la Societe Française d'Hematologie. Nouv Rev Fr Hematol. 1994; 36:353-357, and Isenhart C E, Brandt J T. Platelet aggregation studies for the diagnosis of heparin-induced thrombocytopenia. Am J Clin Pathol. 1993; 99:324-330. A second category of functional assay suitable for use in the present methods for identifying and treating a subject at risk for thrombosis is the serotonin release assay. The serotonin release assay is a well-characterized technique wherein donor platelets are isolated from platelet-rich plasma by differential centrifugation and incubated with radiolabeled serotonin. The platelets then are washed to remove free serotonin. The labeled platelets are then incubated at room temperature with patient serum (heat-treated) in the presence of low (0.1 U/mL) and high (100 U/mL) concentrations of heparin. A positive assay is defined as greater than 20 percent release in the presence of the low concentration of heparin and less than 20 percent release in the presence of the high concentration of heparin. The serotonin release assay is described in further detail in Sheridan D, Carter C, Kelton J G. A diagnostic test for heparin-induced thrombocytopenia. Blood. 1986; 67:27-30, and Favaloro E J, Bernal-Hoyos E, Exner T, Koutts J. Heparin-induced thrombocytopenia: laboratory investigation and confirmation of diagnosis. Pathology. 1992; 24:177-183, both of which are incorporated herein by reference for their description of detection methods.

A third functional assay for use in the present methods for identifying and treating a subject at risk for thrombosis is the heparin-induced platelet agglutination (HIPA) assay is similar to the serotonin release assay in that it uses washed donor platelets and heat-treated patient serum. In contrast to the serotonin release assay, the endpoint for the HIPA assay is macroscopic agglutination of platelets in a microtiter well. This assay is compared to others and methods for its practice are provided in Greinacher A, Michels I, Kiefel V, Mueller-Eckhardt C. A rapid and sensitive test for diagnosing heparin-associated thrombocytopenia. Thromb Haemost. 1991; 66:734-736. Greinacher A, Amiral J, Dummel V, Vissac A, et al. Laboratory diagnosis of heparin-associated thrombocytopenia and comparison of platelet aggregation test, heparin-induced platelet activation test, and platelet factor 4/heparin enzyme-linked immunosorbent assay. Transfusion. 1994; 34:381-385, both references are incorporated herein by reference.

A fourth functional category of functional assay for use in the present methods for identifying and treating a subject at risk for thrombosis is based on flow cytometric techniques. These techniques are based on incubating patient plasma with normal donor platelets in the presence or absence of heparin and then measuring a change in the platelet surface, such as binding of annexin to negatively charged phospholipids or expression of an activation marker such as P-selectin (CD62P). Of particular interest, the assay described by Jy et al. suggests it may be possible to distinguish patients at high risk for thrombosis from other patients with HIT. For this reason, as well as their rapid and efficient through put these assays are particularly useful in the present methods. These techniques are taught by Tomer A. A sensitive and specific functional flow cytometric assay for the diagnosis of heparin-induced thrombocytopenia. Br J Haematol. 1997; 98:648-656; Jy W, et al. A flow cytometric assay of platelet activation marker P-selectin (CD62P) distinguishes heparin-induced thrombocytopenia (HIT) from HIT with thrombosis (HITT). Thromb Haemost. 1999; 82:1255-1259. Tomer A, et al. Determination of heparin-induced thrombocytopenia: a rapid flow cytometric assay for direct demonstration of antibody-mediated platelet activation. Am J Hematol. 1999; 61:53-61. These three references are incorporated herein by reference.

As referenced above, additional laboratory techniques suitable for use in the present methods for identifying and treating a subject at risk for thrombosis are known to those of skill in the art. One group of such examples measure the presence of antibodies capable of binding to PF4 bound to heparin or a heparin-like molecule. The detection system can be modified to detect IgG, IgM, and/or IgA antibodies. Particular examples include those using an enzyme-linked immunosorbent assay (ELISA) format. Examples of antibody detection assays useful in the present methods are taught by Amiral J, Bridey F, Dreyfus M, et al. Platelet factor 4 complexed to heparin is the target for antibodies generated in heparin-induced thrombocytopenia. Thromb Haemost. 1992; 68:95-96; Amiral J, Bridey F, Wolf M, et al. Antibodies to macromolecular platelet factor 4-heparin complexes in heparin-induced thrombocytopenia: a study of 44 cases. Thromb Haemost. 1995; 73:21-28; Visentin G P, Ford S E, Scott J P, Aster R H. Antibodies from patients with heparin-induced thrombocytopenia thrombosis are specific for platelet factor 4 complexed with heparin or bound to endothelial cells. J Clin Invest. 1994; 93:81-88; Amiral J, Wolf M, Fischer A, Boyer-Neumann C, et al. Pathogenicity of IgA and/or IgM antibodies to heparin-PF4 complexes in patients with heparin-induced thrombocytopenia. Br J Haematol. 1996; 92:954-959, each of which are incorporated herein by reference.

Examples of non-invasive monitoring methods suitable for use in the present methods for identifying and treating a subject at risk for thrombosis include those such as ultrasound, venography, imaging, e.g., magnetic resonance or positron emission tomography using a radioactive tracer, and spectroscopic methods (see, Ting Li, Yunglong Sun, Xiao Chen, Yue Zhao, Rongrong Ren, and Mushuang Liu “Non-invasive diagnosis and continuous monitoring of thrombosis in clinics by near-infrared spectroscopy”, Proc. SPIE 9313, Advanced Biomedical and Clinical Diagnostic and Surgical Guidance Systems XIII, 931318 (4 Mar. 2015); https://doi.org/10.1117/12.2081181).

Typically, a combination of the detection and monitoring techniques described above are used to ensure accurate diagnosis of a subject at risk for thrombosis and treatment with the present methods and to ensure proper ongoing care.

In cases of local administration or selective uptake, such as local topical administration, the effective local concentration of fostamatinib may not be related to plasma concentration. Skilled artisans will be able to optimize effective local dosages without undue experimentation.

In the present methods for identifying and treating subjects at risk for thrombosis, subjects typically will receive drugs in addition to fostamatinib or R406. Such additional drugs are known to those of skill in the art of treating thrombosis and maintaining hemostasis in subjects at risk for thrombosis, thrombocytopenia or both. Examples of such additional drugs include, without limitation those listed in Table 1

TABLE 1
Summary of drugs altering hemostasis.

| Class and mechanism of action | Example |
|---|---|
| Anticoagulants | |
| Vitamin K antagonists | Warfarin |
| | Dicumoral |
| Indirect thrombin inhibitors | Heparin |
| | Unfractionated heparin |
| | Low molecular weight heparin |
| | (enoxaparin, dalteparin, tinzaparin |
| Direct thrombin inhibitors | Hirudin derivatives |
| | Desirudin, lepirudin, bivalirudin |
| | Argatroban |
| | Dabigatran |
| Factor Xa inhibitors | Fondaparinux |
| | Rivaroxaban |
| | Apixaban |
| | Edoxaban |
| | Betrixaban (in development) |
| Antiplatelet drugs | |
| COX inhibitors | Aspirin |
| | Nonsteroidal anti-inflammatory drugs |
| Thienopyridine derivatives | Clopidogrel |
| | Ticlopidine |
| | Prasugrel |
| | Ticagrelor |
| Platelet GPIIb/IIIa inhibitors | Abcivimab |
| | Eptifibatide |
| | Tirofiban |
| Thrombolytic/fibrinolytic agents | |
| Tissue plasminogen | Streotokinase |
| | Urokinase |
| Recombinant tissue | Alteplase |
| plasminogen activators | |

COX = Cyclooxygenase,
GPIIb/IIIa = Glycoprotein IIb/IIIa

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration and various factors discussed above. It is contemplated that a typical dosage when used alone or when co-administered with another chemotherapeutic agent will range from about 0.001 mg/kg to about 1000 mg/kg, about 0.01 mg/kg to about 100 mg/kg, or from about 0.1 mg/kg to about 10 mg/kg.

Typical daily administrations of fostamatinib are in the range of 100-300 mg/day, e.g., 100, 150, 200, 250, or 300 mg/day. Administration can be once or twice daily, e.g., 100 or 150 mg BID. Accordingly, pharmaceutical dosage forms comprising a form of fostamatinib may contain from 50-300 mg of a form of fostamatinib, e.g., 50, 100, 150, 200, 250, 300 mg of a form of fostamatinib. In one embodiment, the dosage is 100 mg of a form of fostamatinib (e.g., TAVALISSE®) in tablet form. In another embodiment, the dosage is 150 mg of a form of fostamatinib (e.g., TAVALISSE®) in tablet form.

In one embodiment, the method of the invention if performed by initiating administration of a form of fostamatinib (e.g., TAVALISSE®) at 100 mg orally twice daily with or without food. After 4 weeks, administration is increases to 150 mg twice daily, if needed, to achieve platelet counts of at least 50×109/L as necessary to reduce the risk of bleeding with concomitant inhibition of thrombosis. In one aspect of the disclosed methods for treating thrombosis, subjects are treated with low molecular weight heparin concomitantly with fostamatinib. In another aspect the patients may be treated with warfarin and or coumadin in combination with fostamatinib.

In one embodiment, the amount of a form of fostamatinib in a composition to be administered, or the amount of fostamatinib to be administered in a method of the invention, is a suboptimal dose. As used herein, a suboptimal dose is a dose typically used in a single administration to a patient in monotherapy or in standard of care combination therapies.

The pharmaceutical formulations described hereinabove for use in preventing or treating thrombosis can comprise a form of fostamatinib or R406 as the sole active agent or may further comprise one or more (e.g., two) additional active agents.

Inhibition of Thrombosis In Vivo

Transgenic HIT mice homozygous for both FcγRIIA and human PF4 and null for mouse PF4 are used to evaluate the effects of fostamatinib on immune complexes (IC)-induced thrombocytopenia and thrombosis. 6-12 weeks old transgenic male mice are fed the control chow or chow containing 0.5, 1 and 2 g/kg of R788 for 3 days prior to the injection of either the monoclonal HIT-like antibody KKO or IgG from HIT patients (HIT-IgG). For HIT-induced thrombocytopenia experiments, all mice received heparin subcutaneously at 1600 U/kg body weight once daily for 6 days, starting with first day after antibody injection. Retro-orbital blood collection is performed daily after anesthetizing the animals starting a day before HIT antibody injection to monitor thrombocytopenia by counting platelets using standard equipment.

To assess fostamatinib effect on thrombosis, the mice are fed with drug or vehicle chow followed by injection of the HIT antibodies as described. Next day, each animal is retro-orbitally infused with 1×108 Alexa750-labeled platelets from untreated syngeneic donor mice prior to heparin injection. Animals are terminated 3 hours later, their lungs are excised, perfused with PBS, fixed with 7% paraformaldehyde and imaged using Odyssey Imaging System (LI-COR Bio). Thrombi are counted and scored by intensity using NIS Elements software (Nikon). The results demonstrate that R788 inhibits thrombosis in vivo. For methods, see generally, M. P. Reilly et al. Blood. 2011; 117(7): 2241-2246, Hayes V, Johnston I, Arepally G M, McKenzie S E, Cines D B, Rauova L, Poncz M. Endothelial antigen assembly leads to thrombotic complications in heparin-induced thrombocytopenia. J Clin Invest. 2017 Mar. 1; 127(3):1090-1098 and Rauova L, Zhai L, Kowalska M A, Arepally G M, Cines D B, Poncz M. Role of platelet surface PF4 antigenic complexes in heparin-induced thrombocytopenia pathogenesis: diagnostic and therapeutic implications. Blood. 2006 Mar. 15; 107(6):2346-53. The results demonstrate that Syk inhibition with fostamatinib effectively reduces heparin induced thrombosis.

Inhibition of Deep Vein Thrombosis In Vivo

To assess the effect of fostamatinib on deep vein thrombosis, inferior vena cava (IVC) stenosis model in 8-week old C57Bl/6 WT male mice is used. Mice are fed fostamatinib or vehicle chow for 3 days, anaesthetized using isoflurane and then subjected to laparotomy. After tying off the side branches of the IVC, the IVC is stenosed with a ligature and a 30-gauge spacer to maintain a small degree of blood flow. Mice are allowed to recover, then culled 48 hours after the surgery and the IVC are examined for the presence and size of thrombus. Relative lack of thrombus in fostamatinib chow fed mice demonstrates inhibition of deep vein thrombosis in vivo.

For deep vein thrombosis assay methods generally, see, Low dose Btk inhibitors selectively block platelet activation by CLEC-2. Phillip L. R. Nicolson et al. Haematologica 2020 Jan. 16. pii: haematol. 2019.218545. doi: 10.3324/haematol.2019.218545

Arterial Thrombosis In Vivo

To investigate effect on arterial thrombosis, a ferric chloride injury model in mice is employed. 8-week-old C57Bl6 mice are fed fostamatinib or vehicle chow for 3 days, anaesthetized using mixture of ketamine (100 mg/kg)/xylazine (10 mg/kg) via intraperitoneal injection and have their right jugular vein exposed. Next, rhodamine 6G solution is injected into the right jugular vein to label platelets, exposed carotid artery (CA) staged at the microscope and the thrombosis is induced by applying a piece of filter paper (1×2 mm) saturated with FeCl3 solution directly on the CA. Thrombus formation is identified by accumulation of the fluorescent platelets, which is observed in real-time recording video images under the microscope. Lack of thrombus formation in fostamatinib chow fed mice demonstrates that fostamatinib blocks arterial thrombus formation in vivo.

For arterial thrombosis assay methods generally, see, Wei Li, Thomas M. McIntyre, Roy L. Silverstein. Ferric chloride-induced murine carotid arterial injury: A model of redox pathology. Redox Biology 1 (2013) 50-55, and Wei Li, Marvin Nieman, Anirban Sen Gupta. Ferric Chloride-induced Murine Thrombosis Models. J. Vis. Exp. (115), e54479, doi:10.3791/54479 (2016).

We claim:

1. A method of preventing thrombosis in a subject, the method comprising administering to the subject an effective amount of R406, fostamatinib or a combination thereof effective to prevent thrombosis in the subject.

2. The method according to claim 1, wherein the subject has one or more risk factors for developing thrombosis.

3. The method according to claim 1, wherein the form of fostamatinib is fostamatinib disodium hexahydrate.

4. A method of treating thrombosis in a subject, the method comprising administering to a patient having a thrombus an amount of a form of fostamatinib effective to treat the thrombosis in the patient.

5. The method according to claim 4, wherein the subject has one or more risk factors for developing thrombosis.

6. The method according to claim 4, wherein fostamatinib is administered in the form of fostamatinib disodium hexahydrate.

7. The method according to claim 5, wherein a risk factor is treatment with a TPO agonist.

8. The method according to claim 1, wherein the subject has received heparin.

9. The method according to claim 8, wherein the heparin is high molecular weight heparin.

10. The method according to claim 1, wherein the subject is a perioperative subject.

11. The method according to claim 1, wherein the R406, fostamatinib or a combination thereof is administered in conjunction with heparin.

12. The method according to claim 11, wherein the heparin is low molecular weight heparin.

13. A method for treating or preventing thrombosis in a subject, comprising
identifying a subject at risk for thrombosis; and
administering to the subject an effective amount of R406, fostamatinib or a combination thereof.

14. The method of claim 13, wherein the subject has been treated with a TPO agonist.

15. The method of claim 14, wherein the subject has been treated with romiplostim, eltrombopag, IVIG, or combination thereof.

16. The method of claim 15, wherein the subject is a post-operative subject.

17. The method of claim 16, wherein the subject has been treated with unfractionated heparin.

18. The method of claim 17, further comprising administering low molecular weight heparin.

19. The method of claim 13, wherein identifying a subject at risk for thrombosis comprises in vitro detection of platelet activation.

20. The method of claim 19, wherein in vitro detection of platelet activation comprises a platelet aggregation assay.

21. The method of claim 19, wherein in vitro detection of platelet activation comprises a serotonin release assay.

22. The method of claim 13, wherein identifying a subject at risk for thrombosis comprises detecting heparin-PF4 antibody.

23. The method of claim 14, wherein identifying a subject at risk for thrombosis comprises detecting a greater than 50 percent decrease in the platelet count from baseline.
